(19) United States
(12) Patent Application Publication
(10) Pub. No.: US 2026/0102472 A1
(43) Pub. Date: Apr. 16, 2026

(54) SEMAGLUTIDE IN MEDICAL THERAPY

(71) Applicant: Novo Nordisk A/S, Bagsvaerd (DK)

(72) Inventors: Mikhail Kosiborod, Kansas City, MO (US); Florian Martin Moesgaard Baeres, Dyssegaard (DK); Marie Lindegaard, Koebenhavn S (DK); Anders Gaarsdal Holst, Gentofte (DK)

(21) Appl. No.: 19/422,732

(22) Filed: Dec. 17, 2025

Related U.S. Application Data
(63) Continuation of application No. PCT/US2024/034440, filed on Jun. 18, 2024.
(60) Provisional application No. 63/626,308, filed on Jan. 29, 2024.

(30) Foreign Application Priority Data
Jun. 23, 2023 (EP) 23181162.1

(51) Int. Cl. A61K 38/26; A61K 47/24; A61P 9/04

(52) U.S. Cl. CPC A61K 38/26; A61K 47/24; A61P 9/04

(57) ABSTRACT

The present invention is directed to use of the GLP-1 receptor agonist semaglutide in medical therapy for the treatment of heart failure with preserved ejection fraction (HFpEF).

### CROSS-REFERENCE TO RELATED APPLICATIONS

This application is a continuation of International Application No. PCT/US2024/034440, filed Jun. 18, 2024, which claims priority to European Patent Application 23181162.1, filed Jun. 23, 2023 and U.S. Provisional Application 63/626,308, filed Jan. 29, 2024; all of which are hereby incorporated by reference.

The present invention relates to medical therapy comprising administration of the GLP-1 receptor agonist semaglutide in heart failure with preserved ejection fraction.

### BACKGROUND

Heart failure (HF) is a syndrome caused by impairment of the blood pumping function of the heart. Heart failure with preserved ejection fraction (HFpEF) is one subgroup of HF. The prevalence of HFpEF in humans has increased during the last decades. Heart failure (HF), including HFpEF, remains to be a leading cause of morbidity and mortality. Approximately 50% of patients in the US diagnosed with heart failure (HF) have HFpEF. More than 80% of patients with HFpEF have either overweight or obesity and it is estimated that at least 50% of patients with HFpEF have obesity.

In the semaglutide cardiovascular outcomes trial no reduction was observed for semaglutide compared to placebo with respect to hospitalisation for heart failure where 0.5 mg or 1.0 mg semaglutide was administered subcutaneous once weekly since the outcome was 1.11 (0.77−1.61) [hazard ratio (95% confidence interval)] (Table 2 of Marso et al., N Engl J Med 2016; 375:1834-1844).

SGLT2 inhibitors (empagliflozin and dapagliflozin) have recently been approved for treatment of HF regardless of ejection fraction, but no pharmaceutical therapy specifically targeting the obesity phenotype of HFpEF exists, highlighting a great unmet need (Paulus, N Engl J Med. 2020; 382:679-82).

### SUMMARY

In some embodiments the present invention relates to a method of treatment of heart failure with preserved ejection fraction (HFpEF) comprising administration of semaglutide to a human subject in an amount of at least about 2.4 mg per week.

### BRIEF DESCRIPTION OF DRAWINGS

FIG. 1 shows time from randomisation to first heart failure event leading to either hospitalisation or an urgent visit following administration of semaglutide or placebo.

### DESCRIPTION

In some embodiments the invention relates to a method for the treatment of heart failure with preserved ejection fraction (HFpEF) in a human subject comprising administering semaglutide in an amount of at least about 2.4 mg per week. In some embodiments the invention relates to a method for preventing worsening of or improving heart failure with preserved ejection fraction (HFpEF) in a human subject comprising administration of semaglutide in an amount of at least about 2.4 mg per week. In some embodiments the invention relates to a method for reducing symptoms of heart failure with preserved ejection fraction (HFpEF) in a human subject comprising administration of semaglutide in an amount of at least about 2.4 mg per week.

HFpEF is a form of heart failure where the heart fails to keep up with the circulatory demands of the body or do so at the expense of raised left ventricular filling pressure. HFpEF is characterized by heart failure symptoms (shortness of breath, fatigue, and/or oedema) combined with (i) left ventricular ejection fraction (LVEF) of above 40% and (ii) increased left ventricular filling pressure. LVEF is the ratio of the volume of blood ejected from the left ventricle with each heartbeat divided by the volume of blood contained in the left ventricle when maximally filled. As used herein, the term “increased left ventricular filling pressure” refers to structural and functional changes indicative of left ventricular filling pressure, e.g. measured by cardiac catheterization or estimated by echocardiography. Echocardiographic finding of left ventricular diastolic dysfunction (abnormal relaxation) is also indicative of increased left ventricular filling pressure. In some embodiments the term “HFpEF” as used herein refers to heart failure symptoms (shortness of breath, fatigue, and/or oedema) combined with (i) left ventricular ejection fraction (LVEF) of at least 45% or at least 50% and (ii) increased left ventricular filling pressure. In some embodiments the subject is diagnosed with HFpEF.

In some embodiments the subject has HFpEF and a BMI of at least 30 kg/m², also referred to as obesity-related heart failure with preserved ejection fraction. In some embodiments the subject has a BMI of at least 27 kg/m², which may be referred to as overweight. In some embodiments the subject has a BMI of at least 30 kg/m², which may be referred to as obesity. BMI (body mass index) is a measure of body fat based on height and weight. The formula for calculation is BMI=(weight in kilograms)/(height in meters) 2. In some embodiments the subject has diabetes, such as type 2 diabetes. In some embodiments the subject has a BMI of at least 27 kg/m², such as at least 30 kg/m², and the subject has diabetes, such as type 2 diabetes. In some embodiments the subject has a BMI of at least 30 kg/m² and diabetes. In some embodiments the method provides reduction of body weight.

In some embodiments the invention relates to a method for the treatment of heart failure with preserved ejection fraction (HFpEF) in a human subject and having a BMI of at least 30 kg/m2, wherein the method comprises subcutaneous administration of semaglutide in an amount of at least about 2.4 mg once weekly. In some embodiments the invention relates to a method for the treatment of heart failure with preserved ejection fraction (HFpEF) in a human subject and having a BMI of at least 30 kg/m2, wherein the method comprises subcutaneous administration of semaglutide in an amount of about 2.4 mg to about 10 mg once weekly. In some embodiments the invention relates to a method for the treatment of heart failure with preserved ejection fraction (HFpEF) in a human subject and having a BMI of at least 30 kg/m2, wherein the method comprises subcutaneous administration of semaglutide in an amount of about 2.4 mg once weekly.

In some embodiments the invention relates to a method for preventing worsening of or improving heart failure with preserved ejection fraction (HFpEF) in a human subject and having a BMI of at least 30 kg/m2, wherein the method comprises subcutaneous administration of semaglutide in an amount of at least about 2.4 mg once weekly. In some embodiments the invention relates to a method for preventing worsening of or improving heart failure with preserved ejection fraction (HFpEF) in a human subject and having a BMI of at least 30 kg/m2, wherein the method comprises subcutaneous administration of semaglutide in an amount of about 2.4 mg to about 10 mg once weekly. In some embodiments the invention relates to a method for preventing worsening of or improving heart failure with preserved ejection fraction (HFpEF) in a human subject and having a BMI of at least 30 kg/m2, wherein the method comprises subcutaneous administration of semaglutide in an amount of about 2.4 mg once weekly.

In some embodiments the invention relates to a method for reducing symptoms of heart failure with preserved ejection fraction (HFpEF) in a human subject and having a BMI of at least 30 kg/m2, wherein the method comprises subcutaneous administration of semaglutide in an amount of at least about 2.4 mg once weekly. In some embodiments the invention relates to a method for reducing symptoms of heart failure with preserved ejection fraction (HFpEF) in a human subject and having a BMI of at least 30 kg/m2, wherein the method comprises subcutaneous administration of semaglutide in an amount of about 2.4 mg to about 10 mg once weekly. In some embodiments the invention relates to a method for reducing symptoms of heart failure with preserved ejection fraction (HFpEF) in a human subject and having a BMI of at least 30 kg/m2, wherein the method comprises subcutaneous administration of semaglutide in an amount of about 2.4 mg once weekly.

Patients with heart failure have a significantly poorer health-related quality of life compared to patients with many other common chronic conditions. The Kansas City Cardiomyopathy Questionnaire (KCCQ) is a disease-specific health status instrument composed of 23 items that quantify the domains of physical limitation, symptoms, self-efficacy, social limitation, and health-related quality of life limitation from heart failure (available via cvoutcomes.org). The questions of the KCCQ are as published in the appendix of Green et al., JACC 2000; 35 (5): 1245-55:

| | |
|---|---|
| Question 1 | Heart failure affects different people in different ways. Some feel |
| | shortness of breath while others feel fatigue. Please indicate how |
| | much you are limited by heart failure (shortness of breath or fatigue) in |
| | your ability to do the following activities over the past 2 |
| | weeks. For each of the activities a-f select an answer among: (i) |
| | Extremely Limited, (ii) Quite a bit Limited, (iii) Moderately Limited, (iv) |
| | Slightly Limited, (v) Not at all Limited, and (vi) Limited for other reasons |
| | or did not do the activity: |
| | a: Dressing yourself |
| | b: Showering/Bathing |
| | c: Walking 1 block on level ground |
| | d: Doing yardwork, housework or carrying groceries |
| | e: Climbing a flight of stairs without stopping |
| | f: Hurrying or jogging (as if to catch a bus) |
| Question 2 | Compared with 2 weeks ago, have your symptoms of heart failure |
| | (shortness of breath, fatigue or ankle swelling) changed? Select an |
| | answer among: My symptoms of heart failure have become (i) Much |
| | worse, (ii) Slightly worse, (iii) Not changed, (iv) Slightly better, (v) Much |
| | better, and (vi) I've had no symptoms over the last 2 weeks |
| Question 3 | Over the past 2 weeks, how many times did you have swelling in your |
| | feet, ankles or legs when you woke up in the morning? Select an answer |
| | among: (i) Every morning, (ii) 3 or more times a week, but not every day, |
| | (iii) 1-2 times a week, (iv) Less than once a week, and (v) Never over the |
| | past 2 weeks |
| Question 4 | Over the past 2 weeks, how much has swelling in your feet, ankles or |
| | legs bothered you? Select an answer among: It has been (i) Extremely |
| | bothersome, (ii) Quite a bit bothersome, (iii) Moderately bothersome, (iv) |
| | Slightly bothersome, (v) Not at all bothersome, and (vi) I've had no |
| | swelling |
| Question 5 | Over the past 2 weeks, on average, how many times has fatigue limited |
| | your ability to do what you want? Select an answer among: (i) All of the |
| | time, (ii) Several times per day, (iii) At least once a day, (iv) 3 or more |
| | times per week but not every day, (v) 1-2 times per week, (vi) Less than |
| | once a week, and (vii) Never over the past 2 weeks |
| Question 6 | Over the past 2 weeks, how much has your fatigue bothered you? Select |
| | an answer among: It has been (i) Extremely bothersome, (ii) Quite a bit |
| | bothersome, (iii) Moderately bothersome, (iv) Slightly bothersome, (v) Not |
| | at all bothersome, and (vi) I've had no fatigue |
| Question 7 | Over the past 2 weeks, on average, how many times has shortness of |
| | breath limited your ability to do what you wanted? Select an answer |
| | among: (i) All of the time, (ii) Several times per day, (iii) At least once a |
| | day, (iv) 3 or more times per week but not every day, (v) 1-2 times per |
| | week, (vi) Less than once a week, and (vii) Never over the past 2 weeks |
| Question 8 | Over the past 2 weeks, how much has your shortness of breath |
| | bothered you? It has been (i) Extremely bothersome, (ii) Quite a bit |
| | bothersome, (iii) Moderately bothersome, (iv) Slightly bothersome, (v) Not |
| | at all bothersome, and (vi) I've had no shortness of breath |
| Question 9 | Over the past 2 weeks, on average, how many times have you been |
| | forced to sleep sitting up in a chair or with at least 3 pillows to prop you |
| | up because of shortness of breath? Select an answer among: (i) Every |
| | night, (ii) 3 or more times a week, but not every day, (iii) 1-2 times a |
| | week, (iv) Less than once a week, and (v) Never over the past 2 weeks |
| Question 10 | Heart failure symptoms can worsen for a number of reasons. How sure |
| | are you that you know what to do, or whom to call, if your heart failure |
| | gets worse? Select an answer among: (i) Not at all sure, (ii) esure, (iii) |
| | Somewhat sure, (iv) Mostly sure, and (v) Completely sure |
| Question 11 | How well do you understand what things you are able to do to keep your |
| | heart failure symptoms from getting worse? (for example, weighing |
| | yourself, eating a low salt diet, etc.) |
| | Select an answer among: (i) Do not understand at all, (ii) Do not |
| | understand very well, (iii) Somewhat understand, (iv) Mostly understand, |
| | and (v) Completely understand |
| Question 12 | Over the past 2 weeks, how much has your heart failure limited your |
| | enjoyment of life? Select an answer among: (i) It has extremely limited |
| | my enjoyment of life, (ii) It has limited my enjoyment of life quite a bit, (iii) |
| | It has moderately limited my enjoyment of life, (iv) It has slightly limited |
| | my enjoyment of life, and (v) It has not limited my enjoyment of life at all |
| Question 13 | If you had to spend the rest of your life with your heart failure the way it is |
| | right now, how would you feel about this? Select an answer among: (i) |
| | Not at all satisfied, (ii) Mostly dissatisfied, (iii) Somewhat satisfied, (iv) |
| | Mostly satisfied and, (v) Completely satisfied |
| Question 14 | Over the past 2 weeks, how often have you felt discouraged or down in |
| | the dumps because of your heart failure? Select an answer among: (i) I |
| | felt that way all of the time, (ii) I felt that way most of the time, (iii) I |
| | occasionally felt that way, (iv) I rarely felt that way and, (v) I never felt that |
| | way |
| Question 15 | How much does your heart failure affect your lifestyle? Please indicate |
| | how your heart failure may have limited your participation in the |
| | following activities over the past 2 weeks. For each of the activities g-j |
| | select an answer among: (i) Severely Limited, (ii) Limited quite a bit, (iii) |
| | Moderately Limited, (iv) Slightly Limited, (v) Did not limit at all, and (vi) |
| | Does not apply or did not do for other reasons: |
| | g: Hobbies, recreational activities |
| | h: Working or doing household chores |
| | i: Visiting family or friends out of your home |
| | j: Intimate relationships with loved ones |

The Kansas City Cardiomyopathy Questionnaire clinical summary score (KCCQ-CSS) is used for interpretation of the KCCQ and scores range from 0 to 100 where higher scores indicate better function. KCCQ-CSS is calculated as the mean of the available summary scores Physical Limitation Score and Total Symptom Score from the KCCQ. “Physical Limitation Score” is calculated, if answers to at least three of Questions 1a-f are not missing, as 100*[(mean of Questions 1a-f answered)−1]/4, where answers are scores as follows: Extremely limited=1, Quite a bit limited=2, Moderately limited=3, Slightly limited=4, Not at all limited=5, or Limited for other reasons or did not do=<missing value> (<missing value> will be disregarded in calculations from KCCQ). “Total Symptom Score” is calculated as the mean of the available summary scores Symptom Frequency Score and Symptom Burden Score. “Symptom Frequency Score” is calculated, if answer to at least two of Questions 3, 5, 7 and 9 are not missing, as 100*(mean of S3, S5, S7 and S9 answered), where S3=[(Question 3)−1]/4, S5=[(Question 5)−1]/6, S7=[(Question 7)−1]/6, and S9=[(Question 9)−1]/4; answers to Question 3 are scored as follows: Every morning=1, 3 or more times a week but not every day=2, 1-2 times a week=3, Less than once a week=4, Never over the past 2 weeks=5; answers to Questions 5 and 7 are scored as follows: All of the time=1, Several times a day=2, At least once a day=3, 3 or more times a week but not every day=4, 1-2 times a week=5, Less than once a week=6, or Never over the past 2 weeks=7; and answers to Question 9 are scored as follows: Every night=1, 3 or more times a week but not every day=2, 1-2 times a week=3, Less than once a week=4, or Never over the past 2 weeks=5. “Symptom Burden Score” is calculated, if answer to at least one of Questions 4, 6 and 8 is not missing, as 100*[(mean of Questions 4, 6 and 8 answered)−1]/4 where answers are scored as follows: Extremely bothersome=1, Quite a bit bothersome=2, Moderately bothersome=3, Slightly bothersome=4, Not at all bothersome=5, or I've had no swelling/fatigue/shortness of breath=5. The term “mean of questions answered” as used herein refers to that for a scale with n in questions where the subject must answer m to score the scale, but the subject answers only n-i, where n-i>=m, then the mean of those questions is calculated as (sum of answers to those n-i questions)/(n-i).

In some embodiments the method provides improvement of KKCQ-CSS by at least 5 points. In some embodiments the method provides improvement of KKCQ-CSS by at least 10 points. In some embodiments the method provides improvement of KKCQ-CSS by at least 15 points.

The six minute walk test (6MWT) is a determination of the distance a subject is able to walk over a period of six minutes where the subject traverses, at its own pace and resting as needed, back and forth along a marked walkway of 20 meters with the goal of walking as far as possible without running. The distance obtained from 6MWT is referred to as the six minute walk distance (6MWD). The 6MWT is a direct and timed measure of walking ability, which is technically simple and reproducible.

Heart failure exists in different degrees of severity. The most commonly used classification system of heart failure is the New York Heart Association Functional Classification (also referred to as “NYHA”). NYHA categorises subjects in one of four classes I-IV (Table A), based on their degree of limitation during physical activity, and optionally an additional subgroup A-D based on objective assessments, for further details see The Criteria Committee of the New York Heart Association. Nomenclature and Criteria for Diagnosis of Diseases of the Heart and Great Vessels. 9th ed. Boston, Mass: Little, Brown & Co; 1994:253-256.

TABLE A
NYHA class I-IV criteria

| NYHA | |
|---|---|
| Class | Functional Capacity of the subject |
| I | Subjects with cardiac disease but without resulting limitation of physical activity. |
| | Ordinary physical activity does not cause undue fatigue, palpitation, dyspnoea, |
| | or anginal pain. |
| II | Subjects with cardiac disease resulting in slight limitation of physical activity. |
| | They are comfortable at rest. Ordinary physical activity results in fatigue, |
| | palpitation, dyspnoea, or anginal pain. |
| III | Subjects with cardiac disease resulting in marked limitation of physical activity. |
| | They are comfortable at rest. Less than ordinary activity causes fatigue, |
| | palpitation, dyspnoea, or anginal pain. |
| IV | Subjects with cardiac disease resulting in inability to carry on any physical |
| | activity without discomfort. Symptoms of heart failure or the anginal syndrome |
| | may be present even at rest. If any physical activity is undertaken, discomfort is |
| | increased. |

In some embodiments the subject has heart failure selected from the group consisting of NYHA class II, III and IV. In some embodiments the subject has heart failure NYHA class II. In some embodiments the subject has heart failure NYHA class III. In some embodiments the subject has heart failure NYHA class IV. In some embodiments the method provides an improvement or no worsening of the NYHA class of the subject.

The subject administered semaglutide according to the present invention is a human. In some embodiments the subject is an adult human, e.g. at least 18 years of age.

In some embodiments the method improves physical function. In some embodiments the method improves heart failure symptoms. In some embodiments the method reduces the risk of heart failure symptoms selected from one or more from the group consisting of hospitalisation for heart failure, urgent heart failure visit and CV death. In some embodiments the method reduces the risk of heart failure symptoms selected from the group consisting of hospitalisation for heart failure, urgent heart failure visit and CV death. In some embodiments the method improves physical function, heart failure symptoms and reduces the risk of heart failure symptoms selected from one or more from the group consisting of hospitalisation for heart failure, urgent heart failure visit and CV death. In some embodiments the method improves physical function, heart failure symptoms and reduces the risk of heart failure symptoms selected from the group consisting of hospitalisation for heart failure, urgent heart failure visit and CV death.

In some embodiments the invention relates to a method for the manufacture of a medicament for the treatment of heart failure with preserved ejection fraction (HFpEF) in a human subject comprising administering semaglutide in an amount of at least about 2.4 mg per week. In some embodiments the invention relates to a method for the manufacture of a medicament for the treatment as defined herein.

### Semaglutide

The method of the invention comprises the GLP-1 receptor agonist semaglutide. Semaglutide is N-epsilon26-[2-(2-{2-[2-(2-{2-[(S)-4-Carboxy-4-(17-carboxyheptadecanoyl-amino) butyrylamino]ethoxy}ethoxy) acetylamino]ethoxy}ethoxy) acetyl] [Aib8,Arg34] GLP-1-(7-37) and may be prepared as described in Example 4 of WO2006/097537, the contents of which are incorporated by reference in their entirety. In some embodiments the term “GLP-1 receptor agonist” as used herein refers to any compound, including peptides and non-peptide compounds, which binds to the human GLP-1 receptor with a potency (EC50) of below 100 nM as determined by methods known in the art, see for example WO98/08871, the contents of which are incorporated by reference in their entirety. In some embodiments methods for identifying GLP-1 receptor agonists are described in WO93/19175, the contents of which are incorporated by reference in their entirety.

### Administration Regimen

In some embodiments semaglutide is be administered once daily or less frequent, such as once daily or once weekly. In some embodiments the GLP-1 receptor agonist is administered at any time in the day. In some embodiments the method of the invention is continued for at least about 4 weeks, such as at least about 5 weeks.

In some embodiments the method according to the invention is for chronic management of HFpEF. In some embodiments the term “chronic management” as used herein refers to continuing administering semaglutide according to the method of the invention for an extended period of time, such as at least 1 year or longer. In some embodiments, the term “chronic management” as used herein refers to continuing administering semaglutide according to the method of the invention for at least 5 years, such as at least 10 years. In some embodiments, the term “chronic management” as used herein refers to continuing administering semaglutide according to the method of the invention for at least 15 years or at least 20 years. In some embodiments, the term “chronic management” as used herein refers to continuing administering semaglutide according to the method of the invention for the remaining lifetime of a subject. In some embodiments the method according to the invention is continued for at least about 48 weeks. In some embodiments chronic management comprises administration of semaglutide in an amount and frequency, e.g. as specified herein, sufficient for the treatment of HFpEF.

In some embodiments semaglutide is the sole GLP-1 receptor agonist administered in the methods of the invention. In some embodiments semaglutide is the only active ingredient administered in the methods of the invention. In some embodiments semaglutide is the only active ingredient administered in the methods of the invention. In some embodiments the method of the invention comprises administering one or more additional active ingredients which are not GLP-1 receptor agonists to the subject.

### Parenteral Administration

In some embodiments semaglutide is administered via parenteral administration. In some embodiments parenteral administration is subcutaneous administration, for example subcutaneous injection. In some embodiments semaglutide is administered using a pen-injector, such as a 3 ml disposable pen-injector.

In some embodiments semaglutide is administered in an amount of 0.5-10 mg semaglutide per week, such as 1.0-9.5 mg or 1.5-9.0 mg semaglutide per week. In some embodiments semaglutide is administered in an amount of 2.0-8.5 mg semaglutide per week, such as 2.5-8.0 mg or 3.0-7.5 mg semaglutide per week. In some embodiments semaglutide is administered in an amount of 3.5-7.0 mg semaglutide per week, such as 4.0-6.5 mg or 4.5-5.5 mg semaglutide per week. In some embodiments semaglutide is administered in an amount of about 0.5-5 mg semaglutide per week, such as about 1.0-4.5 mg or about 1.5-4.0 mg or semaglutide per week. In some embodiments semaglutide is administered in an amount of about 2.0-3.5 mg semaglutide per week, such as about 2.5-3.0 mg semaglutide per week. In some embodiments semaglutide is administered in an amount of about 0.7-5 mg semaglutide per week, such as about 0.7-4.5 mg or about 0.7-4.0 mg semaglutide per week. In some embodiments semaglutide is administered in an amount of about 0.7-3.5 mg semaglutide per week, such as about 0.7-3.0 mg or about 0.7-2.5 mg semaglutide per week. In some embodiments semaglutide is administered in an amount of about 2.0-4.0 mg semaglutide per week, such as about 2.0-2.5 mg or about 2.0-3.0 mg semaglutide per week. In some embodiments semaglutide is administered in an amount of about 1.2-3.5 mg semaglutide per week, such as about 1.8-3.2 mg or about 2.4-3.0 mg or semaglutide per week. In some embodiments semaglutide is administered in an amount of about 1.4 mg, such as about 1.5 mg or about 1.6 mg, semaglutide per week. In some embodiments semaglutide is administered in an amount of about 1.7 mg, such as about 1.8 mg or about 1.9 mg, semaglutide per week. In some embodiments semaglutide is administered in an amount of about 2.0 mg, such as about 2.1 mg or about 2.2 mg, semaglutide per week. In some embodiments semaglutide is administered in an amount of about 2.3 mg, such as about 2.4 mg or about 2.5 mg, semaglutide per week. In some embodiments semaglutide is administered in an amount of about 2.6 mg, such as about 2.7 mg or about 2.8 mg, semaglutide per week. In some embodiments semaglutide is administered in an amount of about 2.9 mg, such as about 3.0 mg or about 3.1 mg, semaglutide per week. In some embodiments semaglutide is administered in an amount of about 3.2 mg, such as about 3.3 mg or about 3.4 mg, semaglutide per week.

In some embodiments semaglutide is administered in an amount of about 0.5-5 mg semaglutide once weekly, such as about 1.0-4.5 mg or about 1.5-4.0 mg or semaglutide once weekly. In some embodiments semaglutide is administered in an amount of about 2.0-3.5 mg semaglutide once weekly, such as about 2.5-3.0 mg semaglutide once weekly. In some embodiments semaglutide is administered in an amount of about 0.7-5 mg semaglutide once weekly, such as about 0.7-4.5 mg or about 0.7-4.0 mg semaglutide once weekly. In some embodiments semaglutide is administered in an amount of about 0.7-3.5 mg semaglutide once weekly, such as about 0.7-3.0 mg or about 0.7-2.5 mg semaglutide once weekly. In some embodiments semaglutide is administered in an amount of about 2.0-4.0 mg semaglutide once weekly, such as about 2.0-2.5 mg or about 2.0-3.0 mg semaglutide once weekly. In some embodiments semaglutide is administered in an amount of about 1.2-3.5 mg semaglutide once weekly, such as about 1.8-3.2 mg or about 2.4-3.0 mg or semaglutide once weekly. In some embodiments semaglutide is administered in an amount of about 1.4 mg, such as about 1.5 mg or about 1.6 mg, semaglutide once weekly. In some embodiments semaglutide is administered in an amount of about 1.7 mg, such as about 1.8 mg or about 1.9 mg, semaglutide once weekly. In some embodiments semaglutide is administered in an amount of about 2.0 mg, such as about 2.1 mg or about 2.2 mg, semaglutide once weekly. In some embodiments semaglutide is administered in an amount of about 2.3 mg, such as about 2.4 mg or about 2.5 mg, semaglutide once weekly. In some embodiments semaglutide is administered in an amount of about 2.6 mg, such as about 2.7 mg or about 2.8 mg, semaglutide once weekly. In some embodiments semaglutide is administered in an amount of about 2.9 mg, such as about 3.0 mg or about 3.1 mg, semaglutide once weekly. In some embodiments semaglutide is administered in an amount of about 3.2 mg, such as about 3.3 mg or about 3.4 mg, semaglutide once weekly.

### Oral Administration

In some embodiments semaglutide is administered orally, for example in the form of a solid oral dosage form selected from the group consisting of a tablet, a coated tablet, a sachet and a capsule (such as hard or soft gelatine capsule). In some embodiments semaglutide is administered once daily via oral administration.

In some embodiments semaglutide is administered in an amount of about 5-45 mg, such as about 10-40 mg or about 15-35 mg, via oral administration. In some embodiments semaglutide is administered in an amount of about 20-30 mg via oral administration. In some embodiments semaglutide is administered in an amount of about 7-44 mg, such as about 12-43 mg or about 18-42 mg, via oral administration. In some embodiments semaglutide is administered in an amount of about 20-41 mg, such as about 22-40 mg or about 24-39 mg, via oral administration. In some embodiments semaglutide is administered in an amount of about 26-38 mg, such as about 28-37 mg or about 30-36 mg, via oral administration. In some embodiments semaglutide is administered in an amount of about 30 mg, such as about 31 mg or about 32 mg, via oral administration. In some embodiments semaglutide is administered in an amount of about 33 mg, such as about 34 mg or about 35 mg, via oral administration. In some embodiments semaglutide is administered in an amount of about 36 mg, such as about 37 mg or about 38 mg, via oral administration. In some embodiments semaglutide is administered in an amount of about 39 mg, such as about 40 mg or about 41 mg, via oral administration. In some embodiments semaglutide is administered in an amount of about 3, about 7 or about 14 mg, via oral administration. In some embodiments the treatment dosage for oral administration is in the range of 3-50 mg semaglutide per day, such as 10-40 mg or 15-40 mg, semaglutide per day.

### Dose Escalation

Administration of semaglutide may be initiated via dose escalation, i.e. beginning with an amount lower than the treatment dosage and gradually increasing towards the treatment dosage over time. Dose escalation may help avoid one or more unwanted side effects. As used herein, the term “treatment dosage” refers to the dosage (i.e. amount and administration frequency) of semaglutide. In some embodiments the treatment dosage results in therapeutic effect in the medical indication referred to. In some embodiments the method of the invention comprises an initial step of dose escalation, wherein the subject is administered (i) a dosage of semaglutide in the range of from about one tenth to half of the treatment dosage, and then (ii) every 2-6 weeks, such as about every 2 weeks, about every 4 weeks, or about every 5 weeks, dosage is increased by 1.5-2.5 times, such as about 2 times, until at treatment dosage. In some embodiments the method of the invention comprises an initial step of dose escalation via parenteral administration, wherein the subject is administered (i) a dosage of semaglutide in the range of 0.2-0.5 mg semaglutide per week, such as about 0.35 mg semaglutide per week, and then (ii) every 2-6 weeks, such as about every 4 weeks, dosage is increased by 1.5-2.5 times, such as about 2 times, until at treatment dosage. In some embodiments, and unless otherwise specified, amounts and/or administration frequency for semaglutide mentioned herein refer to the treatment dosage. In some embodiments the treatment dosage for subcutaneous administration is in the range of 0.5-10 mg semaglutide per week, such as 1.0-9.5 mg or 1.5-9.0 mg semaglutide per week. In some embodiments the treatment dosage for subcutaneous administration is in the range of 2.0-8.5 mg semaglutide per week, such as 2.5-8.0 mg or 3.0-7.5 mg semaglutide per week. In some embodiments the treatment dosage for subcutaneous administration is in the range of 3.5-7.0 mg semaglutide per week, such as 4.0-6.5 mg or 4.5-5.5 mg semaglutide per week. In some embodiments the treatment dosage is in the range of 1.2-3.5 mg semaglutide per week, such as 1.8-3.2 mg or 2.4-3.0 mg or semaglutide per week. In some embodiments the treatment dosage is about 2.4 mg semaglutide per week.

### Composition

In the method of the invention semaglutide may be administered in the form of a pharmaceutical composition, also referred to as a composition herein. The pharmaceutical composition may be in a liquid or solid form.

### Parenteral Administration

The pharmaceutical composition may comprise semaglutide in a concentration from 0.1 mg/ml to 100 mg/ml. In some embodiments the pharmaceutical composition comprises 0.01-50 mg/ml, or 0.01-20 mg/ml, or 0.01-10 mg/ml semaglutide. In some embodiments the pharmaceutical composition comprises 0.1-20 mg/ml semaglutide.

The pharmaceutical compositions described herein may further comprise one or more pharmaceutically acceptable excipients, for example selected from the group consisting of buffer system, preservative, tonicity agent, chelating agent, stabilizer and surfactant. In some embodiments the pharmaceutical composition comprises one or more pharmaceutically acceptable excipients, such as one or more selected from the group consisting of a buffer, an isotonic agent, and a preservative. The formulation of pharmaceutically active ingredients with various excipients is known in the art, see e.g. Remington: The Science and Practice of Pharmacy (e.g. 19th edition (1995), and any later editions). The term “excipient” broadly refers to any component other than the active therapeutic ingredient(s), e.g. semaglutide. The excipient may be an inert substance, an inactive substance, and/or a not medicinally active substance.

In some embodiments the pharmaceutical composition has a pH in the range of about 7.0-10.0, such as about 7.0 to about 9.5 or about 7.2 to about 9.5. In some embodiments the pharmaceutical composition has a pH in the range of 7.0-8.5, such as about 7.0 to about 7.8 or about 7.8 to about 8.2. In some embodiments the pharmaceutical composition has a pH of about 7.4. In some embodiments the pharmaceutical composition comprises a phosphate buffer, such as a sodium phosphate buffer, e.g. disodium phosphate. In some embodiments the pharmaceutical composition comprises an isotonic agent, such as propylene glycol or sodium chloride. In some embodiments the pharmaceutical composition comprises a preservative, such as phenol.

The pharmaceutical composition may be in the form of a solution or a suspension. In some embodiments the pharmaceutical composition is aqueous composition, such as an aqueous solution or an aqueous suspension. The term “aqueous composition” is defined as a composition comprising at least 50% (w/w) water. Likewise, the term “aqueous solution” is defined as a solution comprising at least 50% (w/w) water, and the term “aqueous suspension” is defined as a suspension comprising at least 50% (w/w) water. In some embodiments an aqueous composition comprises at least 50% (w/w) water, such as at least 60% (w/w) or at least 70% (w/w) water. In some embodiments an aqueous composition comprises at least 80% (w/w) or at least 90% (w/w) water.

In some embodiments semaglutide is administered in the form of a pharmaceutical composition comprising a phosphate buffer and propylene glycol. In some embodiments semaglutide is administered in the form of a pharmaceutical composition comprising about 2-15 mM phosphate buffer and about 2-25 mg/ml propylene glycol. In some embodiments semaglutide is administered in the form of a pharmaceutical composition comprising about 0.1-20 mg/ml semaglutide, about 2-15 mM phosphate buffer, about 2-25 mg/ml propylene glycol, and a pH in the range of about 7.0-9.0. In some embodiments semaglutide is administered in the form of a pharmaceutical composition comprising about 0.1-20 mg/ml semaglutide, about 2-15 mM phosphate buffer, about 2-25 mg/ml propylene glycol, about 1-18 mg/ml phenol, and a pH in the range of about 7.0-9.0. In some embodiments semaglutide is administered in the form of a pharmaceutical composition comprising about 1.0 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 0.5-4 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 0.5-1.5 mg/ml semaglutide, about 1.42 mg/ml disodium phosphate dihydrate, about 14.0 mg/ml propylene glycol, about 5.5 mg/ml phenol, and a pH of about 7.4. In some embodiments semaglutide is administered in the form of a pharmaceutical composition comprising about 1.0-3.5 mg/ml semaglutide, about 1.42 mg/ml disodium phosphate dihydrate, about 14.0 mg/ml propylene glycol, about 5.5 mg/ml phenol, and a pH of about 7.4.

In some embodiments semaglutide is administered in the form of a pharmaceutical composition comprising a phosphate buffer and sodium chloride. In some embodiments semaglutide is administered in the form of a pharmaceutical composition comprising about 2-15 mM phosphate buffer and about 2-25 mg/ml sodium chloride. In some embodiments semaglutide is administered in the form of a pharmaceutical composition comprising about 0.1-20 mg/ml semaglutide, about 2-15 mM phosphate buffer, about 2-25 mg/ml sodium chloride, and a pH in the range of about 7.0-9.0. In some embodiments semaglutide is administered in the form of a pharmaceutical composition comprising about 0.1-20 mg/ml semaglutide, about 2-15 mM phosphate buffer, about 2-25 mg/ml sodium chloride, about 1-18 mg/ml phenol, and a pH in the range of about 7.0-9.0. In some embodiments semaglutide is administered in the form of a pharmaceutical composition comprising about 1.0 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 0.5-4 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 0.5-1.5 mg/ml semaglutide, about 1.42 mg/ml disodium phosphate dihydrate, about 14.0 mg/ml sodium chloride, about 5.5 mg/ml phenol, and a pH of about 7.4. In some embodiments semaglutide is administered in the form of a pharmaceutical composition comprising about 1.0-3.5 mg/ml semaglutide, about 1.42 mg/ml disodium phosphate dihydrate, about 14.0 mg/ml sodium chloride, about 5.5 mg/ml phenol, and a pH of about 7.4.

### Oral Administration

In the method of the invention semaglutide may be administered in the form of a solid composition via oral administration. The solid composition may be suitable for administration by the oral route, e.g. as described further herein. In some embodiments the solid composition comprises at least one pharmaceutically acceptable excipient. The term “excipient” as used herein broadly refers to any component other than the active therapeutic ingredient(s) or active pharmaceutical ingredient(s) (API(s)). The excipient may be a pharmaceutically inert substance, an inactive substance, and/or a therapeutically or medicinally inactive substance. For oral administration, the excipient may serve various purposes, e.g. as a carrier, vehicle, filler, binder, lubricant, glidant, disintegrant, flow control agent, crystallization inhibitors solubilizer, stabilizer, colouring agent, flavouring agent, surfactant, emulsifier or combinations of thereof and/or to improve administration, and/or absorption of the therapeutically active substance(s) or active pharmaceutical ingredient(s). The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013). In some embodiments the excipients for oral administration may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization inhibitors such as Povidone, etc.; solubilizers such as Pluronic, Povidone, etc.; colouring agents, including dyes and pigments such as iron oxide red or yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In some embodiments the solid composition comprises a binder, such as povidone; starches; celluloses and derivatives thereof, such as microcrystalline cellulose, e.g., Avicel PH from FMC (Philadelphia, PA), hydroxypropyl cellulose hydroxylethyl cellulose and hydroxylpropylmethyl cellulose METHOCEL from Dow Chemical Corp. (Midland, MI); sucrose; dextrose; corn syrup; polysaccharides; and gelatin. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the solid composition comprises Avicel, such as Aavicel PH 102. Suitable binders for wet granulation or dry granulation are corn starch, polyvinyl pyrrolidone (povidon), vinylpyrrolidone-vinylacetate copolymer (copovidone) and cellulose derivatives like hydroxymethylcellulose, hydroxyethylcellulose, hydroxypropylcellulose and hydroxyl-propylmethylcellulose. In some embodiments the solid composition comprises povidone.

In some embodiments the solid composition comprises a filler which may be selected from lactose, mannitol, erythritol, sucrose, sorbitol, calcium phosphate, such as calciumhydrogen phosphate, microcrystalline cellulose, powdered cellulose, confectioner's sugar, compressible sugar, dextrates, dextrin and dextrose. In some embodiments the solid composition comprises microcrystalline cellulose, such as Avicel PH 102 or Avicel PH 200. In some embodiments the solid composition comprises a lubricant and/or a glidant.

In some embodiments the composition comprises a lubricant and/or a glidant, such as talc, magnesium stearate, calcium stearate, zinc stearate, glyceryl behenate, glyceryl debehenate, behenoyl polyoxyl-8 glycerides, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol etc. In some embodiments the solid composition comprises magnesium stearate or glyceryl debehenate (such as the product Compritol® 888 ATO).

In some embodiments the solid composition comprises a disintegrant, such as sodium starch glycolate, polacrilin potassium, sodium starch glycolate, crospovidon, croscarmellose, sodium carboxymethylcellulose or dried corn starch. The solid composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, non-ionic surfactants, and/or zwitterionic surfactants.

In some embodiments the solid composition further comprises a delivery agent or absorption enhancer is for the present invention an excipient capable of increasing the oral exposure of semaglutide. The delivery agent may be a salt of N-(8-(2-hydroxybenzoyl)amino) caprylic acid (also referred to herein as a salt of NAC), which contains the anion N-(8-(2-hydroxybenzoyl)amino) caprylate. The structural formula of N-(8-(2-hydroxybenzoyl)amino) caprylate is shown in Formula (II).

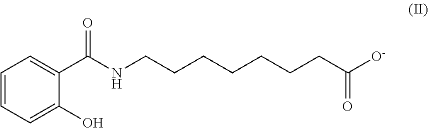 [SMILES: O=C([O-])CCCCCCCNC(=O)c1ccccc1O]

In some embodiments the salt of N-(8-(2-hydroxybenzoyl)amino) caprylic acid comprises one monovalent cation, two monovalent cations or one divalent cation. In some embodiments the salt of N-(8-(2-hydroxybenzoyl)amino) caprylic acid is selected from the group consisting of the sodium salt, potassium salt and/or calcium salt of N-(8-(2-hydroxybenzoyl)amino) caprylic acid. In some embodiments the salt of N-(8-(2-hydroxybenzoyl)amino) caprylic acid is selected from the group consisting of the sodium salt, potassium salt and/or the ammonium salt. In some embodiments the salt of N-(8-(2-hydroxybenzoyl)amino) caprylic acid is the sodium salt or the potassium salt. Salts of N-(8-(2-hydroxybenzoyl)amino) caprylate may be prepared using the method described in e.g. WO96/030036, WO00/046182, WO01/092206 or WO2008/028859, the contents of which are incorporated by reference in their entirety. The salt of N-(8-(2-hydroxybenzoyl)amino) caprylic acid may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino) caprylic acid as well as combinations thereof. In some embodiments the delivery agent is a salt of N-(8-(2-hydroxybenzoyl)amino) caprylic acid as described in WO2007/121318, the contents of which are incorporated by reference in their entirety. In some embodiments the delivery agent is sodium N-(8-(2-hydroxybenzoyl)amino) caprylate (referred to as “SNAC” herein), also known as sodium 8-(salicyloylamino) octanoate.

In some embodiments the composition for use in the invention is in the form of a solid composition, such as a tablet, for oral administration. In some embodiments the solid composition comprises semaglutide in an amount in the range of 0.1-50 mg, such as 0.5 to 40 mg or 1-30 mg. In some embodiments the solid composition comprises semaglutide in an amount in the range of 2-20 mg, such as 3-18 mg or 5-15 mg. In some embodiments the solid composition comprises semaglutide in an amount of about 3 mg, such as about 7 mg or about 14 mg.

In some embodiments least 30% (w/w) of the solid composition (e.g. tablet) is a salt of N-(8-(2-hydroxybenzoyl)amino) caprylic acid. In some embodiments least 50% (w/w) of the solid composition (e.g. tablet) is a salt of N-(8-(2-hydroxybenzoyl)amino) caprylic acid. In some embodiments the amount of the salt of N-(8-(2-hydroxybenzoyl)amino) caprylic acid per dose unit of the composition is in the range of 0.20-0.5 mmol, 0.25-1.0 mmol, 0.30-0.75 mmol, or such as 0.45-0.65 mmol. In some embodiments the amount of SNAC in the composition is in the range of 75-600 mg. In some embodiments the amount of SNAC in the composition is in the range of 75-400 mg, such as from 80-350 mg, such as from about 100 to about 300 mg per dose unit.

In some embodiments the solid composition comprises a lubricant, such as magnesium stearate. In some embodiments a unit dose of the solid composition comprises: 0.1-50 mg semaglutide, 25-600 mg salt of N-(8-(2-hydroxybenzoyl)amino) caprylic acid (NAC) (such as the sodium salt of NAC (SNAC)), and 0-25 mg lubricant.

In some embodiments the solid composition comprises a) 0.5-50 mg semaglutide, b)

20-800 mg, such as 50-500 mg, of a salt of N-(8-(2-hydroxybenzoyl)amino) caprylic acid and c) 0.6-20 mg, such as 1-10 mg, lubricant, wherein said salt of N-(8-(2-hydroxybenzoyl)amino) caprylic acid constitutes at least 90% (w/w), such as at least 95% (w/w), of the excipients of the composition.

In some embodiments the solid composition the solid composition is in the form of a dose unit, such as tablet. In some embodiments the weight of the unit dose is in the range of 50 mg to 1000 mg, such as in the range of 50-750 mg, or such as about 100-500 mg. In some embodiments the weight of the dose unit is in the range of 75 mg to 350 mg, such as in the range of 50-300 mg or 100-400 mg. The tablet may comprise 30% (w/w) salt of N-(8-(2-hydroxybenzoyl)amino) caprylic acid, such as the sodium salt of N-(8-(2-hydroxybenzoyl)amino) caprylic acid (SNAC). In some embodiments the composition may be granulated prior to being compressed to tablets. The composition may comprise a granular part and/or an extra-granular part, wherein the granular part has been granulated and the extra-granular part has been added after granulation. Semaglutide may be included in the granular part or the extra-granular part. In some embodiments the extra-granular part comprises semaglutide. In an embodiment the extra-granular part may further comprise a lubricant and/or a glidant. In an embodiment the granular part may comprise a lubricant and/or a glidant. In an embodiment the granular part and the extra-granular part comprise a lubricant and/or a glidant.

Unless otherwise stated, ranges herein include their end points. In some embodiments the term “a” means “one or more”. In some embodiments, and unless otherwise indicated herein, terms presented in singular form also include the plural situation. Herein the term “about” means±10% of the value referred to, and includes the value. In some embodiments the term “comprise” as used herein includes “consist of”. In some embodiments, pH values referred to herein are measured at 25° C.

### Non-Limiting Embodiments of the Invention

The invention is further described by the following non-limiting embodiments:
1. A method for the treatment of heart failure with preserved ejection fraction (HFpEF) in a human subject comprising administering semaglutide in an amount of at least about 2.4 mg per week.
2. A method for the treatment of heart failure with preserved ejection fraction (HFpEF) in a human subject and having a BMI of at least 30 kg/m², wherein said method comprises subcutaneous administration of semaglutide in an amount of at least about 2.4 mg once weekly.
3. A method for the treatment of heart failure with preserved ejection fraction (HFpEF) in a human subject and having a BMI of at least 30 kg/m², wherein said method comprises subcutaneous administration of semaglutide in an amount of about 2.4 mg to about 10 mg once weekly.
4. A method for the treatment of heart failure with preserved ejection fraction (HFpEF) in a human subject and having a BMI of at least 30 kg/m², wherein said method comprises subcutaneous administration of semaglutide in an amount of about 2.4 mg once weekly.
5. A method for preventing worsening of or improving heart failure with preserved ejection fraction (HFpEF) in a human subject comprising administration of semaglutide in an amount of at least about 2.4 mg per week.
6. A method for preventing worsening of or improving heart failure with preserved ejection fraction (HFpEF) in a human subject and having a BMI of at least 30 kg/m², wherein said method comprises subcutaneous administration of semaglutide in an amount of at least about 2.4 mg once weekly.
7. A method for preventing worsening of or improving heart failure with preserved ejection fraction (HFpEF) in a human subject and having a BMI of at least 30 kg/m², wherein said method comprises subcutaneous administration of semaglutide in an amount of about 2.4 mg to about 10 mg once weekly.
8. A method for preventing worsening of or improving heart failure with preserved ejection fraction (HFpEF) in a human subject and having a BMI of at least 30 kg/m², wherein said method comprises subcutaneous administration of semaglutide in an amount of about 2.4 mg once weekly.
9. A method for reducing symptoms of heart failure with preserved ejection fraction (HFpEF) in a human subject comprising administration of semaglutide in an amount of at least about 2.4 mg per week.
10. A method for reducing symptoms of heart failure with preserved ejection fraction (HFpEF) in a human subject and having a BMI of at least 30 kg/m², wherein said method comprises subcutaneous administration of semaglutide in an amount of at least about 2.4 mg once weekly.
11. A method for reducing symptoms of heart failure with preserved ejection fraction (HFpEF) in a human subject and having a BMI of at least 30 kg/m², wherein said method comprises subcutaneous administration of semaglutide in an amount of about 2.4 mg to about 10 mg once weekly.
12. A method for reducing symptoms of heart failure with preserved ejection fraction (HFpEF) in a human subject and having a BMI of at least 30 kg/m², wherein said method comprises subcutaneous administration of semaglutide in an amount of about 2.4 mg once weekly.
13. The method according to any one of the preceding embodiments, wherein said subject has a BMI of at least 27 kg/m².
14. The method according to any one of the preceding embodiments, wherein said subject has a BMI of at least 30 kg/m².
15. The method according to any one of the preceding embodiments, wherein said subject has heart failure selected from the group consisting of NYHA class II, III and IV.
16. The method according to any one of the preceding embodiments, wherein said method provides an improvement or no worsening of the NYHA class of said subject.
17. The method according to any one of the preceding embodiments, wherein said subject has diabetes.
18. The method according to any one of the preceding embodiments, wherein said subject has a BMI of at least 27 kg/m², such as at least 30 kg/m², and said subject has diabetes.
19. The method according to any one of the preceding embodiments, wherein said subject has a BMI of at least 30 kg/m² and diabetes.
20. The method according to any one of the preceding embodiments, wherein said subject is diagnosed with HFpEF.
21. The method according to any one of the preceding embodiments, wherein said method provides improvement of KKCQ-CSS by at least 5 points.
22. The method according to any one of the preceding embodiments, wherein said method provides improvement of KKCQ-CSS by at least 10 points.
23. The method according to any one of the preceding embodiments, wherein said method provides improvement of KKCQ-CSS by at least 15 points.
24. The method according to any one of the preceding embodiments, wherein said method provides reduction of body weight.
25. The method according to any one of the preceding embodiments, wherein said semaglutide is administered by parenteral administration.
26. The method according to any one of the preceding embodiments, wherein said semaglutide is administered by subcutaneous administration.
27. The method according to any one of the preceding embodiments, wherein said semaglutide is administered in an amount of about 2.4 mg to about 10 mg per week.
28. The method according to any one of the preceding embodiments, wherein said semaglutide is administered in an amount of about 2.4 mg to about 10 mg per week.
29. The method according to any one of the preceding embodiments, wherein said semaglutide is administered in an amount of about 2.4 mg per week.
30. The method according to any one of the preceding embodiments, wherein said semaglutide is administered once daily or less frequently.
31. The method according to any one of the preceding embodiments, wherein said semaglutide is administered once weekly.
32. The method according to any one of the preceding embodiments, wherein said semaglutide is administered in an amount of about 2.4 mg to about 10 mg once weekly.
33. The method according to any one of the preceding embodiments, wherein said semaglutide is administered in an amount of about 2.4 mg once weekly.
34. The method according to any one of the preceding embodiments, wherein said semaglutide is administered subcutaneously.
35. The method according to any one of the preceding embodiments, wherein said semaglutide is administered for a period of at least 4 weeks.
36. The method according to any one of the preceding embodiments, wherein said semaglutide is administered for a period of at least 5 weeks.
37. The method according to any one of the preceding embodiments, wherein said method is for chronic management of HFpEF.
38. The method according to any one of the preceding embodiments, wherein said semaglutide is the sole active ingredient administered to said subject.
39. The method according to any one of the preceding embodiments, wherein said subject is administered one or more additional active ingredients which are not GLP-1 receptor agonists.
40. The method according to any one of the preceding embodiments, wherein said semaglutide is administered in the form of a pharmaceutical composition comprising one or more pharmaceutically acceptable excipients.
41. The method according to any one of the preceding embodiments, wherein said semaglutide is the sole active ingredient in said pharmaceutical composition.
42. The method according to any one of the preceding embodiments, wherein said pharmaceutical composition comprises one or more additional active ingredients which are not GLP-1 receptor agonists.
43. The method according to any one of the preceding embodiments, wherein said pharmaceutical composition is in the form of an aqueous solution.
44. The method according to any one of the preceding embodiments, wherein said pharmaceutical composition comprises 0.5-5.0 mg/ml semaglutide.
45. The method according to any one of the preceding embodiments, wherein said pharmaceutical composition has a pH in the range of about 7.2 to about 7.6, such as about 7.4.
46. The method according to any one of the preceding embodiments, wherein said pharmaceutical composition further comprises a phosphate buffer and propylene glycol.
47. The method according to any one of the preceding embodiments, wherein said pharmaceutical composition further comprises about 2-15 mM phosphate buffer and about 2-25 mg/ml propylene glycol.
48. The method according to any one of the preceding embodiments, wherein said pharmaceutical composition is an aqueous solution comprising about 1.0 mg/ml semaglutide, about 1.42 mg/ml disodium hydrogen phosphate dihydrate, about 14.0 mg/ml propylene glycol, about 5.50 mg/ml phenol at about pH 7.4.
49. The method according to any one of embodiments 1-45, wherein said pharmaceutical composition further comprises about 2-15 mM phosphate buffer and about 2-25 mg/ml sodium chloride.
50. The method according to any one of embodiments 1-45, wherein said pharmaceutical composition is an aqueous solution comprising about 1.0 mg/ml semaglutide, about 1.42 mg/ml disodium hydrogen phosphate dihydrate, about 14.0 mg/ml sodium chloride, about 5.50 mg/ml phenol at about pH 7.4.
51. The method according to any one of the preceding embodiments, wherein said semaglutide is administered by oral administration.
52. The method according to any one of the preceding embodiments, wherein said semaglutide is administered once daily by oral administration.
53. The method according to any one of the preceding embodiments, wherein said semaglutide is administered in an amount of 5-45 mg semaglutide once daily by oral administration.
54. The method according to any one of the preceding embodiments, wherein said semaglutide is administered by oral administration in the form of a tablet.
55. The method according to any one of the preceding embodiments, wherein said semaglutide is administered by oral administration in the form of a solid composition, such as a tablet.
56. The method according to any one of the preceding embodiments, wherein said semaglutide is administered by oral administration in the form of a solid composition comprising SNAC, such as at least 30% (w/w) SNAC.
57. The method according to any one of the preceding embodiments, wherein said method improves physical function.
58. The method according to any one of the preceding embodiments, wherein said method improves heart failure symptoms.
59. The method according to any one of the preceding embodiments, wherein said method reduces the risk of heart failure symptoms selected from one or more from the group consisting of hospitalisation for heart failure, urgent heart failure visit and CV death.
60. The method according to any one of the preceding embodiments, wherein said method reduces the risk of heart failure symptoms selected from the group consisting of hospitalisation for heart failure, urgent heart failure visit and CV death.
61. The method according to any one of the preceding embodiments, wherein said method improves physical function, heart failure symptoms and reduces the risk of heart failure symptoms selected from one or more from the group consisting of hospitalisation for heart failure, urgent heart failure visit and CV death.
62. The method according to any one of the preceding embodiments, wherein said method improves physical function, heart failure symptoms and reduces the risk of heart failure symptoms selected from the group consisting of hospitalisation for heart failure, urgent heart failure visit and CV death.

### EXAMPLES

### Example 1: Semaglutide in Subjects with Obesity-Related Heart Failure with Preserved Ejection Fraction

A randomised, double-blind, international multicentre, placebo-controlled clinical trial with semaglutide was carried out with 529 human subjects with obesity-related heart failure with preserved ejection fraction. Key inclusion criteria were body mass index (BMI) of at least 30 kg/m², NYHA Class II-IV, and LVEF of at least 45% at time of screening for the clinical trial. Key exclusion criteria were self-reported change in body weight of more than 5 kg within 90 days before screening for the clinical trial, diabetes, or HbA1c of at least 6.5%. Detailed inclusion and exclusion criteria are listed in Table 1.

Subjects were randomised in a 1:1 manner to receive either 2.4 mg semaglutide or placebo once weekly subcutaneously as add-on to standard of care. Baseline characteristics of randomised subjects are shown in Table 2. After randomization, a 16-week dose escalation period followed and then a 36-week maintenance period. Subjects administered semaglutide initiated treatment with a once-weekly dose of 0.25 mg semaglutide and followed a fixed dose escalation regimen, with dose increase every 4 weeks (through dosages of 0.5, 1.0, and 1.7 mg semaglutide once weekly), until the 2.4 mg semaglutide once weekly maintenance dose was reached after 16 weeks. The 0.25 mg, 0.5 mg or 1 mg dosages of semaglutide were given in a 0.5 mL single-dose pen and the 1.7 or 2.4 mg dosages of semaglutide were given in a 0.75 mL single-dose pen. The composition administered was an aqueous solution comprising semaglutide, 1.42 mg/ml disodium phosphate dihydrate, 8.25 mg/ml sodium chloride at pH of approximately 7.4.

TABLE 1
Inclusion and exclusion criteria

| | Definition | |
|---|---|---|
| Inclusion | Subjects were eligible for the trial only if all the following criteria | |
| criteria | applied: | |
| | [symbol] | Male or female, age ≥18 years |
| | [symbol] | BMI ≥ 30.0 kg/m² |
| | [symbol] | NYHA Class II-IV |
| | [symbol] | LVEF ≥45% at screening |
| | [symbol] | No hospitalisations due to HF between screening and |
| | | randomisation |
| | [symbol] | Able to perform the 6MWT at screening with a minimum distance of |
| | | 100 metres |
| | [symbol] | KCCQ clinical summary score <90 at screening |
| | [symbol] | At least one of the following: |
| | | Mean PWP ≥15 mm Hg or LVEDP ≥25 mm Hg documented |
| | | during catheterization at rest; or PA diastolic pressure |
| | | measured by implantable monitor ≥15 mm Hg or PWP; or |
| | | LVEDP ≥25 mm Hg documented during catheterization at |
| | | exercise |
| | | Elevated NT-proBNP^{a, b} at screening combined with ≥1 of the |
| | | following: (i) septal é <7 cm/sec or lateral é <10 cm/sec or |
| | | average E/é ≥15; (ii) PA systolic pressure >35 mm Hg, (iii) LA |
| | | enlargement^{c}, (iv) LV hypertrophy with septal thickness or |
| | | posterior wall thickness ≥1.2 cm |
| | | Hospitalization with a primary diagnosis of decompensated HF |
| | | requiring IV loop diuretic treatment within the previous 12 |
| | | months combined with ≥2 of the following: (i) septal é <7 |
| | | cm/sec or lateral é <10 cm/sec or average E/é ≥15; (ii) PA |
| | | systolic pressure >35 mm Hg, (iii) LA enlargement^{c}, (iv) LV |
| | | hypertrophy with septal thickness or posterior wall |
| | | thickness ≥1.2 cm, (v) ongoing use of diuretic therapy for ≥30 days |
| | | before screening |
| Exclusion | | Subjects were excluded from the trial if any of the following criteria |
| criteria | | applied: MI, stroke, hospitalization for HF, unstable angina pectoris, or TIA |
| | | within 30 days prior to the day of screening. SBP >160 mm Hg at |
| | | screening. Planned coronary, carotid or peripheral artery revascularization. |
| | | Any other condition judged by the investigator to be the primary cause of |
| | | dyspnea. Bariatric surgery prior to screening or planned within the trial |
| | | time course. Self-reported change in body weight >5 kg within 90 days |
| | | before screening. HbA1c ≥6.5% (48 mmol/mol) based on latest available |
| | | value from medical records, no older than 3 months or if unavailable at |
| | | local measurement at screening. History of T1D or T2D (history of |
| | | gestational diabetes allowed). Treatment with any GLP-1 RA within 90 |
| | | days prior to the day of screening. Personal or first-degree relative(s) |
| | | history of MEN2 or MTC. Acute pancreatitis within the last 180 days prior |
| | | to screening or history or presence of chronic pancreatitis. End-stage renal |
| | | disease or chronic or intermittent hemodialysis or peritoneal dialysis. |

Echocardiographic features were required documented within 12 months of screening.
^{a}Analyzed by the central laboratory.
^{b}≥220 pg/mL (for patients with BMI <35.0 and sinus rhythm), ≥660 pg/mL (for patients with BMI <35.0 and persistent/permanent atrial fibrillation (AF)), ≥125 pg/mL (for patients with BMI ≥35.0 and sinus rhythm), or ≥375 pg/mL (for patients with BMI ≥35.0 and persistent/permanent AF).
^{c}Width ≥3.8 cm or length ≥5.0 cm or area ≥20.0 cm² or volume ≥55 mL or volume index ≥29 mL/m².
6MWT: 6-minute walk test. BMI: body mass index. GLP-1 RA: glucagon like peptide-1 receptor agonist. HF: heart failure. HFpEF: heart failure with preserved ejection fraction. KCCQ: Kansas City Cardiomyopathy Questionnaire. LA: left atrial. LV: left ventricular. LVEDP: left ventricular end diastolic pressure. LVEF: left ventricular ejection fraction. MEN2: multiple endocrine neoplasia type 2. MI: myocardial infarction. NT-proBNP: N-terminal pro-brain type natriuretic peptide. MTC: medullary thyroid carcinoma. NYHA: New York Heart Association. PA: pulmonary artery. PWP: pulmonary wedge pressure. SBP: systolic blood pressure. T1D: type 1 diabetes. T2D: type 2 diabetes. TIA: transient ischemic attack.

TABLE 2
Baseline characteristics of randomised subjects

| | Total | |
|---|---|---|
| | (N = 529) | |
| Female, n (%) | 297 | (56.1) |
| Age, years | 69 | (62; 75) |
| Ethnicity | | |
| Hispanic or Latino | 36 | (6.8) |
| Not Hispanic or Latino | 493 | (93.2) |
| Race | | |
| Asian | — | |
| Black or African American | 21 | (4.0) |
| Other | 1 | (0.2) |
| White | 507 | (95.8) |
| Body weight, kg | 105.1 | (92.4; 120.8) |
| BMI, kg/m² | 37.0 | (33.7; 41.4) |
| Waist circumference, cm | 119.4 | (110.5; 128.0) |
| SBP, mm Hg | 133 | (121; 144) |
| NT-proBNP, pg/mL | 450.6 | (218.2; 1015) |
| LVEF, % | 57.0 | (50.0; 60.0) |
| KCCQ-CSS | 58.9 | (41.7; 72.9) |
| 6MWT, meters | 320.0 | (240.0; 389.0) |
| Comorbidities at screening, n (%) | | |
| Atrial fibrillation | 275 | (52.0) |
| Hypertension | 433 | (81.9) |
| Coronary artery disease | 180 | (34.0) |
| Obstructive sleep apnea | 65 | (12.3) |
| NYHA class, n (%) | | |
| Class II | 350 | (66.2) |
| Class III-IV | 179 | (33.8) |

Data are median (IQR), unless otherwise indicated and are from the full analysis set.
IQR: Interquartile range.

### Results

Results from the clinical trial are presented in Tables 3a-8 and are based on all randomised subjects with all available assessments included regardless of treatment status. The KCCQ clinical summary score (KCCQ-CSS) was calculated as described herein. The six minutes walk distance (6MWD) was determined as described herein using the 6MWT. Heart failure leading to hospitalisation or an urgent visit were defined as a new episode or worsening of existing heart failure leading to an urgent, unscheduled hospital admission or clinic/office/emergency department visit. Estimated treatment differences relative to placebo and two-sided p-values were obtained from an ANCOVA with missing data multiple imputed (from retrieved drop-outs within treatment) and single imputation with an unfavourable value for those subjects with missing data due to cardiovascular death or having a prior HF event. The win-ratio for the hierarchical endpoint was estimated by pair-wise comparisons between subjects from each treatment group to establish a win or loss for each of the endpoints using a similar imputation approach for missing data. CI: Confidence interval.

TABLE 3a
Observed KCCQ-CSS mean change from baseline to week 52

| Time since randomisation (weeks) | Semaglutide 2.4 mg | Placebo |
|---|---|---|
| 0 | 0 | 0 |
| 20 | 11.0 | 8.1 |
| 36 | 15.0 | 9.1 |
| 52 | 16.8 | 10.3 |

TABLE 3b
KCCQ-CSS change from baseline to week 52

| Change from baseline | | | |
|---|---|---|---|
| to week 52 (score) | Estimate | 95% CI | p-value |
| Semaglutide 2.4 mg | 16.6 | | |
| Placebo | 8.7 | | |
| Treatment difference | | | |
| Semaglutide 2.4 mg vs. placebo | 7.8 | 4.8 to 10.9 | <0.0001 |

TABLE 3c
Odds of improving at least 5 points
from baseline KCCQ-CSS at week 52

| Improvement of ≥5 points in KCCQ-CSS | | |
|---|---|---|
| from baseline to week 52 | Estimate | 95% CI |
| Semaglutide 2.4 mg* | 75% | |
| Placebo* | 64% | |
| Treatment Odds ratio | | |
| Semaglutide 2.4 mg vs. placebo | 1.88 | 1.26 to 2.79 |

*Observed proportion improving ≥5 points from baseline

TABLE 3d
Odds of improving at least 10 points
from baseline KCCQ-CSS at week 52

| Improvement of ≥10 points in KCCQ-CSS | | |
|---|---|---|
| from baseline to week 52 | Estimate | 95% CI |
| Semaglutide 2.4 mg* | 63% | |
| Placebo* | 49% | |
| Treatment Odds ratio | | |
| Semaglutide 2.4 mg vs. placebo | 2.10 | 1.43 to 3.07 |

*Observed proportion improving ≥10 points from baseline

TABLE 4a
Observed body weight (%) mean change from baseline to week 52

| Time since randomisation (weeks) | Semaglutide 2.4 mg | Placebo |
|---|---|---|
| 0 | 0 | 0 |
| 4 | −2.1 | −0.7 |
| 8 | −3.2 | −1.1 |
| 12 | −5.0 | −1.3 |
| 16 | −6.6 | −1.5 |
| 20 | −7.8 | −1.8 |
| 28 | −10.2 | −2.0 |
| 36 | −12.1 | −2.2 |
| 44 | −12.9 | −2.3 |
| 52 | −13.9 | −2.5 |

TABLE 4b
Body weight (%) change from baseline to week 52

| Change from baseline | | | |
|---|---|---|---|
| to week 52 (%) | Estimate | 95% CI | p-value |
| Semaglutide 2.4 mg | −13.3 | | |
| Placebo | −2.6 | | |
| Treatment difference | | | |
| Semaglutide 2.4 mg vs. placebo | −10.7 | −11.9 to −9.4 | <0.0001 |

TABLE 5a
Observed six minutes walk distance (meters)
mean change from baseline to week

| Time since randomisation (weeks) | Semaglutide 2.4 mg | Placebo |
|---|---|---|
| 0 | 0 | 0 |
| 20 | 14.3 | 5.4 |
| 52 | 23.5 | 5.8 |

TABLE 5b
Six minutes walk distance (meters)
change from baseline to week 52

| Change from baseline | | | |
|---|---|---|---|
| to week 52 (meters) | Estimate | 95% CI | p-value |
| Semaglutide 2.4 mg | 21.5 | | |
| Placebo | 1.2 | | |
| Treatment difference | | | |
| Semaglutide 2.4 mg vs. placebo | 20.3 | 8.6 to 32.1 | 0.0007 |

TABLE 6
The hierarchical composite endpoint (win ratio)

| | Wins: Semaglutide | Loss: Semaglutide |
|---|---|---|
| Endpoint | 2.4 mg | 2.4 mg |
| All-cause death | 1.5% | 1.1% |
| Number of HF events | 3.3% | 0.0% |
| Time-to-first HF event | 0.0% | 0.0% |
| Change in KCCQ-CSS ≥15 points difference | 33.7% | 17.5% |
| Change in KCCQ-CSS ≥10 points difference | 7.2% | 5.6% |
| Change in KCCQ-CSS ≥5 points difference | 8.2% | 7.0% |
| Change in 6MWD ≥30 meters difference | 6.3% | 3.7% |
| Total | 60.1% | 34.9% |
| Stratified Win-Ratio | 1.72 95% CI 1.37 to 2.15 p < 0.0001 | |
| (Semaglutide 2.4 mg/placebo) | | |

Proportion of ties: 5.1%.

TABLE 7
Improvement or worsening in NYHA
class from baseline to week 52

| | Improvement | No change | Worsening |
|---|---|---|---|
| Semaglutide 2.4 mg | 36.4% | 61.7% | 2.0% |
| Placebo | 27.2% | 68.3% | 4.5% |

TABLE 8
Time to first HF event from randomisation leading
to either hospitalisation or an urgent visit

| Hazard ratio | Estimate | 95% CI |
|---|---|---|
| Semaglutide 2.4 mg/placebo | 0.08 | 0.00 to 0.42 |

The results in Tables 3a and 3b show that patients treated with semaglutide achieved a significantly larger improvement in KCCQ-CSS from baseline to 52 weeks compared with placebo. The results in Table 3c show that the odds of achieving an improvement in KCCQ-CSS of 5 point or more was 1.88 times greater with semaglutide compared with placebo. The results in Table 3d show that the odds of achieving an improvement in KCCQ-CSS of 10 point or more was 2.10 times greater with semaglutide compared with placebo.

The results in Tables 4a and 4b show that patients treated with semaglutide achieved a significantly larger body weight loss from baseline to 52 weeks compared with placebo.

The results in Tables 5a and 5b show that patients treated with semaglutide achieved a significantly larger improvement in six minutes walk distance (6MWD) from baseline to 52 weeks compared with placebo.

The results in Table 6 show that treatment with semaglutide resulted in a greater proportion of wins as there was 72% more wins on semaglutide as compared to placebo.

The wins favoured semaglutide for all key components of the hierarchical composite endpoint, with treatment difference of 15 points or more in the KCCQ-CSS contributed to the most wins for semaglutide.

The results in Table 7 show that more patients treated with semaglutide improved or observed no change in NYHA class from baseline to 52 weeks compared with placebo.

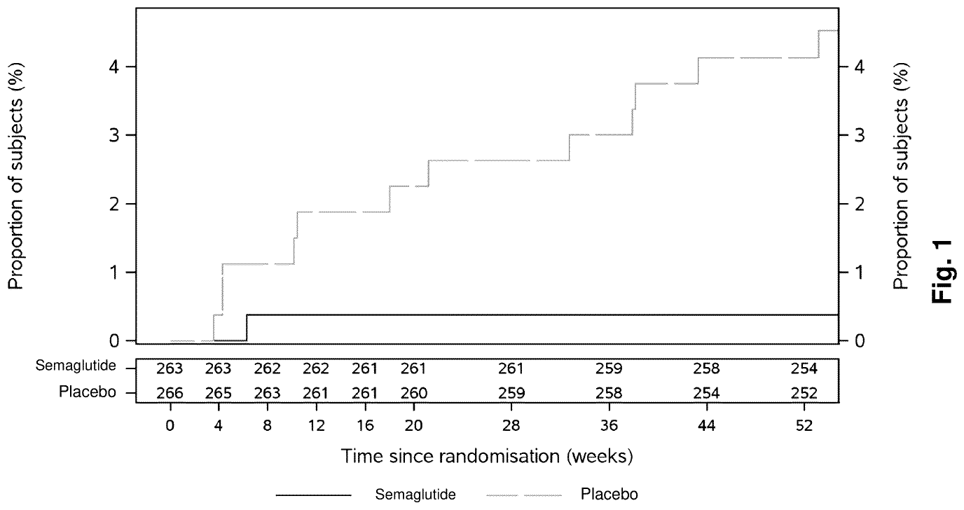

The results in Table 8 show that the risk of experiencing a heart failure event was less with semaglutide compared to placebo. Results of time from randomisation to first heart failure event leading to either hospitalisation or an urgent visit are shown in FIG. 1.

These results show that semaglutide was confirmed superior as compared to placebo for the endpoints of change in KCCQ-CSS, change in body weight, change in 6MWD and hierarchical composite endpoint. The results for the endpoints of patients achieving ≥5 or ≥10 points improvement in KCCQ-CSS, improvement/deterioration in NYHA class and time to first HF event presented herein support the positive effect of semaglutide in this population with heart failure and preserved ejection fraction and obesity.

Thus, in patients with heart failure and preserved ejection fraction and obesity, semaglutide showed clinically relevant, substantial and sustained improvements in symptoms, physical limitations and exercise function together with substantial and sustained weight loss and reduced inflammation

### Example 2: Semaglutide in Subjects with Obesity-Related Heart Failure with Preserved Ejection Fraction, and Type 2 Diabetes

A randomised, double-blind, international multicentre, placebo-controlled clinical trial with semaglutide was carried out with 616 human subjects with obesity-related heart failure with preserved ejection fraction, and type 2 diabetes.

Key inclusion criteria were body mass index (BMI) of at least 30 kg/m², NYHA Class II-IV, LVEF of at least 45% at time of screening for the clinical trial, diagnosis of type 2 diabetes at least 90 days prior to the day of screening for the clinical trial, and HbAlc of no more than 10.0% as measured at the screening visit. Key exclusion criteria were self-reported change in body weight of more than 5 kg within 90 days before screening for the clinical trial or uncontrolled and potentially unstable diabetic retinopathy or maculopathy. Absence of diabetic retinopathy or maculopathy was verified by a fundus examination. Detailed inclusion and exclusion criteria are listed in Table 9.

Subjects were randomised in a 1:1 manner to receive either 2.4 mg semaglutide or placebo once weekly as add-on to standard of care. After randomization, a 16-week dose escalation period followed and then a 36-week maintenance period. Subjects administered semaglutide initiated treatment with a once-weekly dose of 0.25 mg and followed a fixed dose escalation regimen, with dose increase every 4 weeks (through dosages of 0.5, 1.0, 1.7 mg once weekly), until the 2.4 mg once weekly maintenance dose was reached after 16 weeks.

TABLE 9
Inclusion and exclusion criteria

| | Definition |
|---|---|
| Inclusion | Subjects were eligible to be included in the trial only if all of the following |
| criteria | criteria applied: |
| | Male or female, age ≥18 years |
| | BMI ≥30.0 kg/m² |
| | NYHA Class II-IV |
| | LVEF ≥45% at screening |
| | No hospitalisations due to heart failure between screening and |
| | randomisation |
| | Able to perform the 6MWT at screening with a minimum distance of 100 |
| | metres |
| | KCCQ clinical summary score <90 at screening |
| | At least one of the following: |
| | (i) Mean PWP ≥15 mmHg or LVEDP ≥15 mmHg documented |
| | during catheterisation at rest; (ii)PA diastolic pressure measured |
| | by implantable monitor ≥15 mmHg; or (iii) PWP or LVEDP ≥25 |
| | mmHg documented during catheterisation at exercise |
| | Elevated NT-proBNP^{a, b} at screening in combination with ≥1 of the |
| | following: (i) Septal é <7 cm/sec or lateral é <10 cm/sec or |
| | average E/é ≥15, (ii) PA systolic pressure >35 mmHg, (iii) Left |
| | atrial (LA) enlargement^{c}, (iv) LV hypertrophy with septal thickness |
| | or posterior wall thickness ≥1.2 cm |
| | Hospitalisation with a primary diagnosis of decompensated heart |
| | failure requiring intravenous loop diuretic treatment within the |
| | previous 12 months in combination with ≥2 of the following: (i) |
| | septal é <7 cm/sec or lateral é <10 cm/sec or average E/é ≥15, |
| | (ii) PA systolic pressure >35 mmHg, (iii) LA enlargement^{c}, (iv) LV |
| | hypertrophy with septal thickness or posterior wall thickness ≥1.2 cm, |
| | (v) ongoing use of diuretic therapy for at least 30 days prior |
| | to screening |
| | Diagnosed with T2D ≥90 days prior to screening |
| | Subject treated with diet, exercise, and/or glucose lowering |
| | treatment* according to local label in stable dosing for at least |
| | 30 days prior to screening (*: OAD(s): unchanged drug(s), dose |
| | and dosing frequency, Insulin(s): unchanged regimen (basal, |
| | basal + bolus, premix combination) with stable total daily insulin |
| | dose as judged by the investigator) |
| | HbA_{1c} of ≤10.0% as measured at the screening visit |
| Exclusion | Subjects were excluded from the trial if any of the following criteria applied: |
| criteria | MI, stroke, hospitalisation for heart failure, unstable angina pectoris or TIA |
| | within 30 days prior to the day of screening. SBP >160 mmHg at screening. |
| | Planned coronary, carotid or peripheral artery revascularization. Any other |
| | condition judged by the investigator to be the primary cause of dyspnoea. |
| | Bariatric surgery prior to screening or planned bariatric surgery within the trial |
| | time course. Self-reported change in body weight >5 kg within 90 days before |
| | screening. History of T1D (history of gestational diabetes is allowed). |
| | Treatment with any GLP-1 receptor agonist within 90 days prior to the day of |
| | screening. Uncontrolled and potentially unstable diabetic retinopathy or |
| | maculopathy (verified by a fundus examination performed within 90 days |
| | prior to screening or in the period between screening and randomisation; |
| | pharmacological pupil-dilation required unless using a digital fundus |
| | photography camera specified for non-dilated examination). Recurrent severe |
| | hypoglycaemic episodes within the last year as judged by the investigator. |
| | Treatment with continuous subcutaneous insulin infusion. Personal or first- |
| | degree relative(s) history of MEN2 or MTC. Acute pancreatitis within the last |
| | 180 days prior to screening or history or presence of chronic pancreatitis. |
| | End-stage renal disease or chronic or intermittent haemodialysis or peritoneal |
| | dialysis. Presence or history of malignant neoplasm within 5 years prior to the |
| | day of screening. |

Echocardiographic features were required documented within 12 months of screening.
^{a}Analyzed by the central laboratory.
^{b}≥220 pg/mL (for patients with BMI <35.0 and sinus rhythm), ≥660 pg/mL (for patients with BMI <35.0 and persistent/permanent atrial fibrillation (AF)), ≥125 pg/mL (for patients with BMI ≥35.0 and sinus rhythm), or ≥375 pg/mL (for patients with BMI ≥35.0 and persistent/permanent AF).
^{c}Width ≥3.8 cm or length ≥5.0 cm or area ≥20.0 cm² or volume ≥55 mL or volume index ≥29 mL/m².
6MWT: 6-minute walk test. BMI: body mass index. GLP-1 RA: glucagon like peptide-1 receptor agonist. HbA_{1c}: haemoglobin A1c. HF: heart failure. HFpEF: heart failure with preserved ejection fraction. KCCQ: Kansas City Cardiomyopathy Questionnaire. LA: left atrial. LV: left ventricular. LVEDP: left ventricular end diastolic pressure. LVEF: left ventricular ejection fraction. MEN2: multiple endocrine neoplasia type 2. MI: myocardial infarction. NT-proBNP: N-terminal pro-brain type natriuretic peptide. MTC: medullary thyroid carcinoma. NYHA: New York Heart Association. OAD: Oral antidiabetic drug (such as metformin). PA: pulmonary artery. PWP: pulmonary wedge pressure. SBP: systolic blood pressure. T1D: type 1 diabetes. T2D: type 2 diabetes. TIA: transient ischemic attack.

Baseline characteristics of randomised subjects are shown in in Table 10.

TABLE 10
Baseline characteristics of randomised subjects

Total
(N = 616)*

| | | |
|---|---|---|
| Female, n (%) | 273 | (44.3) |
| Age, years | 69 | (63; 75) |
| Ethnicity | | |
| Hispanic or Latino | 76 | (12.3) |
| Not Hispanic or Latino | 540 | (87.7) |
| Race | | |
| Asian | 76 | (12.3) |
| Black or African American | 18 | (2.9) |
| Other | 3 | (0.5) |
| White | 519 | (84.3) |
| Body weight, kg | 102.7 | (90.5; 117.7) |
| BMI, kg/m² | 36.9 | (33.5; 41.3) |
| Waist circumference, cm | 120.3 | (112.0; 130.0) |
| SBP, mm Hg | 135 | (125; 144) |
| NT-proBNP, pg/mL | 493.0 | (245.7; 1018.6) |
| LVEF, % | 56.0 | (50.0; 60.0) |
| KCCQ-CSS | 59.4 | (43.8; 72.0) |
| 6MWT, meters | 280.0 | (203.5; 350.0) |
| HbA1c, % | 6.8 | (6.2; 7.5) |
| eGFR (CKD-EPI), mL/min/1.73 m² | 69.0 | (50.0; 87.5) |
| Diabetes duration, years | 8.0 | (3.9; 14.8) |
| Comorbidities at screening, n (%) | | |
| Atrial fibrillation | 243 | (39.4) |
| Hypertension | 526 | (85.4) |
| Coronary artery disease | 273 | (44.3) |
| Obstructive sleep apnea | 53 | (8.6) |
| NYHA class, n (%) | | |
| Class II | 435 | (70.6) |
| Class III-IV | 181 | (29.3) |

Data are median (IQR), unless otherwise indicated, and are from the full analysis set. IQR: Interquartile range.
*617 subjects were randomised but one subject was excluded from the full analysis set. eGFR (CKD-EPI): estimated glomerular filtration rate calculated based on the creatinine value using the CKD-EPI equation.

### Results

Results from the clinical trial are presented in Tables 11a-16 and are based on all randomised subjects with all available assessments included regardless of treatment status. The KCCQ clinical summary score (KCCQ-CSS) was calculated as described herein. The six minutes walk distance (6MWD) was determined as described herein using the 6MWT. Heart failure leading to hospitalisation or an urgent visit were defined as a new episode or worsening of existing heart failure leading to an urgent, unscheduled hospital admission or clinic/office/emergency department visit. Estimated treatment differences relative to placebo and two-sided p-values were obtained from an ANCOVA with missing data multiple imputed (from retrieved drop-outs within treatment) and single imputation with an unfavourable value for those subjects with missing data due to cardiovascular death or having a prior HF event. The win-ratio for the hierarchical endpoint was estimated by pair-wise comparisons between subjects from each treatment group to establish a win or loss for each of the endpoints using a similar imputation approach for missing data. CI: Confidence interval.

TABLE 11a
Observed KCCQ-CSS mean change from baseline to week 52

| Time since randomisation (weeks) | Semaglutide 2.4 mg | Placebo |
|---|---|---|
| 0 | 0 | 0 |
| 20 | 11.1 | 8.3 |
| 36 | 14.2 | 8.8 |
| 52 | 14.4 | 7.6 |

TABLE 11b
KCCQ-CSS change from baseline to week 52

| | Change from baseline to week 52 (score) | | |
|---|---|---|---|
| | Estimate | 95% CI | p-value |
| Semaglutide 2.4 mg | 13.7 | | |
| Placebo | 6.4 | | |
| Treatment difference | | | |
| Semaglutide 2.4 mg vs. | 7.3 | [4.1; 10.4] | <0.0001 |
| placebo | | | |

TABLE 11c
Odds of improving at least 5 points
from baseline KCCQ-CSS at week 52

| | Improvement of ≥5 points in KCCQ-CSS | |
|---|---|---|
| | from baseline to week 52 | |
| | Estimate | 95% CI |
| Semaglutide 2.4 mg* | 73% | |
| Placebo* | 55% | |
| Treatment Odds ratio | | |
| Semaglutide 2.4 mg vs. | 2.29 | 1.60 to 3.28 |
| placebo | | |

*Observed proportion improving ≥5 points from baseline

TABLE 11d
Odds of improving at least 10 points
from baseline KCCQ-CSS at week 52

| | Improvement of ≥10 points in KCCQ-CSS | |
|---|---|---|
| | from baseline to week 52 | |
| | Estimate | 95% CI |
| Semaglutide 2.4 mg* | 58% | |
| Placebo* | 43% | |
| Treatment Odds ratio | | |
| Semaglutide 2.4 mg vs. | 2.06 | 1.45 to 2.92 |
| placebo | | |

*Observed proportion improving ≥10 points from baseline

TABLE 12a
Observed body weight (%) mean change from baseline to week 52

| Time since randomisation | Semaglutide | |
|---|---|---|
| (weeks) | 2.4 mg | Placebo |
| 0 | 0 | 0 |
| 4 | −1.8 | −0.6 |
| 8 | −3.1 | −1.1 |
| 12 | −4.5 | −1.7 |
| 16 | −5.8 | −1.8 |
| 20 | −6.6 | −2.3 |
| 28 | −8.0 | −2.5 |
| 36 | −9.1 | −2.8 |
| 44 | −9.8 | −2.9 |
| 52 | −10.2 | −3.2 |

TABLE 12b
Body weight (%) change from baseline to week 52

| Change from baseline to week | | | |
|---|---|---|---|
| 52 (%) | Estimate | 95% CI | p-value |
| Semaglutide 2.4 mg | −9.8 | | |
| Placebo | −3.4 | | |
| Treatment difference | | | |
| Semaglutide 2.4 mg vs. placebo | −6.4 | −7.6 to −5.2 | <0.0001 |

TABLE 13a
Observed six minutes walk distance (meters)
mean change from baseline to week 52

| Time since randomisation | Semaglutide | |
|---|---|---|
| (weeks) | 2.4 mg | Placebo |
| 0 | 0 | 0 |
| 20 | 23.0 | 4.7 |
| 52 | 18.6 | 5.0 |

TABLE 13b
Six minutes walk distance (meters)
change from baseline to week 52

| | Change from baseline to week 52 (meters) | | |
|---|---|---|---|
| | Estimate | 95% CI | p-value |
| Semaglutide 2.4 mg | 12.7 | | |
| Placebo | −1.6 | | |
| Treatment difference | | | |
| Semaglutide 2.4 mg vs. | 14.3 | 3.7 to 24.9 | 0.0084 |
| placebo | | | |

TABLE 14
The hierarchical composite endpoint (win ratio)

| | Wins: Semaglutide | Loss: Semaglutide |
|---|---|---|
| Endpoint | 2.4 mg | 2.4 mg |
| All-cause death | 2.9% | 1.9% |
| Number of HF events | 4.7% | 1.2% |
| Time-to-first HF event | 0.0% | 0.0% |
| Change in KCCQ-CSS ≥15 | 32.3% | 18.2% |
| points difference | | |
| Change in KCCQ-CSS ≥10 | 6.6% | 5.0% |
| points difference | | |
| Change in KCCQ-CSS ≥5 | 7.3% | 6.2% |
| points difference | | |
| Change in 6MWD ≥30 | 4.9% | 4.1% |
| meters difference | | |
| Total | 58.7% | 36.8% |

| | |
|---|---|
| Stratified Win-Ratio | 1.58 95% CI 1.29 to 1.94 <0.0001 |
| (Semaglutide 2.4 mg/placebo) | |

Proportion of ties: 4.5%

TABLE 15
Improvement or worsening in NYHA
class from baseline to week 52

| | Improvement | No change | Worsening |
|---|---|---|---|
| Semaglutide 2.4 mg | 33.9% | 63.7% | 2.4% |
| Placebo | 22.5% | 71.0% | 6.5% |

TABLE 16
Time to first HF event from randomisation leading
to either hospitalisation or an urgent visit

| Hazard ratio | Estimate | 95% CI |
|---|---|---|
| Semaglutide 2.4 mg/placebo | 0.40 | 0.15 to 0.92 |

The results in Tables 11a and 11b show that patients treated with semaglutide achieved a significantly larger improvement in KCCQ-CSS from baseline to 52 weeks compared with placebo.

The results in Table 11c show that the odds of achieving an improvement in KCCQ-CSS of 5 point or more was 2.29 times greater with semaglutide compared with placebo.

The results in Table 11d show that the odds of achieving an improvement in KCCQ-CSS of 10 point or more was 2.06 times greater with semaglutide compared with placebo.

The results in Tables 12a and 12b show that patients treated with semaglutide achieved a significantly larger body weight loss from baseline to 52 weeks compared with placebo.

The results in Tables 13a and 13b show that patients treated with semaglutide achieved a significantly larger improvement in six minutes walk distance (6MWD) from baseline to 52 weeks compared with placebo.

The results in Table 14 show that treatment with semaglutide resulted in a greater proportion of wins as there was 59% more wins on semaglutide as compared to placebo. The wins favoured semaglutide for all key components of the hierarchical composite endpoint, with treatment difference of 15 points or more in the KCCQ-CSS contributed to the most wins for semaglutide.

The results in Table 15 show that more patients treated with semaglutide improved or observed no change in NYHA class from baseline to 52 weeks compared with placebo.

The results in Table 16 show that the risk of experiencing a heart failure event was less with semaglutide compared to placebo.

These results show that semaglutide was confirmed superior as compared to placebo for the endpoints of change in KCCQ-CSS, change in body weight, change in 6MWD and hierarchical composite endpoint. The results for the endpoints of patients achieving ≥5 or ≥10 points improvement in KCCQ-CSS, improvement/deterioration in NYHA class and time to first HF event presented herein support the positive effect of semaglutide in this population with heart failure and preserved ejection fraction and obesity.

In patients with heart failure and preserved ejection fraction, obesity and type 2 diabetes, semaglutide showed clinically relevant, substantial and sustained improvements in symptoms, physical limitations and exercise function together with substantial and sustained weight loss and reduced inflammation.

### Example 3: Semaglutide in Subjects with Established Cardiovascular Disease and Obesity or Overweight

A randomised, double-blind, international multicentre, placebo-controlled clinical trial with semaglutide was carried out with 17604 human subjects with established cardiovascular (CV) disease and obesity or overweight.

Key inclusion criteria were at least 45 years of age, BMI of at least 27 kg/m² and established (CV) disease. Key exclusion criteria were type 1 diabetes or type 2 diabetes. Detailed inclusion and exclusion criteria are listed in Table 17.

Subjects were randomised in a 1:1 manner to receive either 2.4 mg semaglutide or placebo once weekly as add-on to standard of care. After randomization, a 16-week dose escalation period followed and then an event driven maintenance period with a mean (+SD) duration of exposure to semaglutide or placebo in the overall trial population of 34.2 (±13.7) months (33.3 (±14.4) months for semaglutide and 35.1 (±13.0) months for placebo). Subjects administered semaglutide initiated treatment with a once-weekly dose of 0.25 mg and followed a fixed dose escalation regimen, with dose increase every 4 weeks (through dosages of 0.5, 1.0, 1.7 mg once weekly), until the 2.4 mg once weekly maintenance dose was reached after 16 weeks.

TABLE 17
Inclusion and exclusion criteria

| | Definition |
|---|---|
| Inclusion | Subjects were eligible for the trial only if all the following criteria applied: |
| criteria | Informed consent obtained before any trial-related activities. Trial- |
| | related activities are any procedures that are carried out as part of the |
| | trial, including activities to determine suitability for the trial |
| | Male or female, age ≥45 years at the time of signing informed consent |
| | Body mass index (BMI) ≥27 kg/m² |
| | Established CV disease as evidenced by at least one of the following: |
| | a) prior myocardial infarction; b) prior stroke (ischemic or hemorrhagic |
| | stroke); or c) symptomatic peripheral arterial disease (PAD), as |
| | evidenced by intermittent claudication with ankle-brachial index |
| | (ABI) <0.85 (at rest), or peripheral arterial revascularization procedure, or |
| | amputation due to atherosclerotic disease |
| Exclusion | Subjects were excluded from the trial if any of the following criteria |
| criteria | applied: Myocardial infarction, stroke, hospitalisation for unstable angina |
| | pectoris or transient ischaemic attack within the past 60 days prior to the |
| | day of screening. Planned coronary, carotid or peripheral artery |
| | revascularisation known on the day of screening. Presently classified as |
| | being in New York Heart Association (NYHA) Class IV heart failure. |
| | HbA1c ≥48 mmol/mol (6.5%) as measured by the central laboratory at |
| | screening. History of type 1 diabetes or type 2 diabetes (history of |
| | gestational diabetes is allowed). Treatment with glucose-lowering agent(s) |
| | within 90 days before screening. Treatment with any GLP-1 RA within 90 |
| | days before screening. History or presence of chronic pancreatitis. |
| | Presence of acute pancreatitis within the past 180 days prior to the day of |
| | screening. Personal or first degree relative(s) history of multiple endocrine |
| | neoplasia type 2 or medullary thyroid carcinoma. End stage renal disease |
| | or chronic or intermittent haemodialysis or peritoneal dialysis. Presence or |
| | history of malignant neoplasms within the past 5 years prior to the day of |
| | screening. Basal and squamous cell skin cancer and any carcinoma in- |
| | situ are allowed. Severe psychiatric disorder which in the investigator's |
| | opinion could compromise compliance with the protocol. Known or |
| | suspected hypersensitivity to trial product(s) or related products. Previous |
| | participation in this trial. Participation is defined as randomisation. Receipt |
| | of any investigational medicinal product within 30 days before screening. |
| | Female who is pregnant, breast-feeding or intends to become pregnant or |
| | is of child-bearing potential and not using a highly effective contraceptive |
| | method. Any disorder, unwillingness or inability, which in the investigator's |
| | opinion, might jeopardise the subject's safety or compliance with the |
| | protocol. |

Baseline characteristics of randomised subjects are shown in in Table 18.

TABLE 18
Baseline characteristics of randomised subjects

| | Total (N = 17604) |
|---|---|
| Female, n (%) | 4872(27.7) |
| Age, years | 61.6 (8.9) |
| Ethnicity n (%) | |
| Hispanic or Latino | 1822(10.3) |
| Not Hispanic or Latino | 15611(88.7) |
| Not reported | 171(1.0) |
| Race n(%) | |
| Asian | 1447(8.2) |
| Black or African American | 671(3.8) |
| White | 14791(84.0) |
| Native Hawaiian or Other Pacific Islander | 8(<0.1) |
| American Indian or Alaska Native | 44(0.2) |
| Other | 474(2.7) |
| Not reported | 169(1.0) |
| Body weight, kg | 96.7(17.7) |
| BMI, kg/m² | 33.3(5.0) |
| Waist circumference, cm | 111.3 (13.1) |
| SBP, mm Hg | 131(15.4) |
| HbA1c, % | 5.8(0.3) |
| eGFR (CKD-EPI), mL/min/1.73 m² | 82.5(17.4) |
| hsCRP ≥2 mg/dL n(%) | 8246 (46.8) |
| Comorbidities at screening, n (%) | |
| Atrial fibrillation | 1554 (8.8) |
| Hypertension | 14392 (81.8) |
| Coronary heart disease | 14452 (82.1) |
| Obstructive sleep apnea | 2550 (14.5) |
| Chronic heart failure | 4286 (24.3) |

Data are mean (standard deviation), unless otherwise indicated, and are from the full analysis set. eGFR (CKD-EPI): estimated glomerular filtration rate calculated based on the creatinine value using the CKD-EPI equation. SBP: systolic blood pressure. eGFR (CKD-EPI): estimated glomerular filtration rate calculated based on the creatinine value using the CKD-EPI equation. hsCRP: C-reactive protein measured in the high-sensitivity C-reactive protein assay.

### Results

Results from the clinical trial are presented in Tables 19-20 based on 8803 and 8801 subjects randomised to semaglutide and placebo, respectively. Results from the chronic heart failure (HF) population of the clinical trial are presented in Tables 21-22 based on 2155 and 2131 chronic HF subjects randomised to semaglutide and placebo, respectively. Heart failure leading to hospitalisation was defined as hospital admission for at least 24 hours with a primary diagnosis of heart failure with documented new or worsening symptoms due to heart failure; and objective evidence of new or worsening heart failure (physical findings and/or laboratory evidence or invasive diagnostic evidence); and increased/initiated appropriate treatment. Estimated hazard ratios relative to placebo and two-sided p-values were obtained from a Cox proportional hazards model with randomised treatment as a fixed factor. CI: Confidence interval.

TABLE 19
Heart failure leading to hospitalisation from baseline
to end-of-trial (mean duration 39.8 months)

| | Time since randomisation to end-of-trial | | |
|---|---|---|---|
| | | | Two-sided |
| | Events (%) | 95% CI | p-value |
| Semaglutide 2.4 mg | 97 (1.1) | | |
| Placebo | 122 (1.4) | | |
| Treatment Hazard ratio | | | |
| Semaglutide 2.4 mg vs. placebo | 0.79 | [0.60; 1.03] | 0.08 |

TABLE 20
CV death from baseline to end-of-
trial (mean duration: 39.8 months)

| | Time since randomisation to end-of-trial | | |
|---|---|---|---|
| | | | Two-sided |
| | Events (%) | 95% CI | p-value |
| Semaglutide 2.4 mg | 223 (2.5) | | |
| Placebo | 262 (3.0) | | |
| Treatment Hazard ratio | | | |
| Semaglutide 2.4 mg vs. placebo | 0.85 | [0.71-1.01] | 0.07 |

TABLE 21
Heart failure leading to hospitalisation from baseline to end-of-
trial (mean duration 39.8 months) - subjects with chronic HF only

| | Time since randomisation to end-of-trial | | |
|---|---|---|---|
| | | | Two-sided |
| | Events (%) | 95% CI | p-value |
| Semaglutide 2.4 mg | 61 (2.8) | | |
| Placebo | 69 (3.2) | | |
| Treatment Hazard ratio | | | |
| Semaglutide 2.4 mg vs. placebo | 0.86 | (0.61-1.21) | 0.38 |

TABLE 22
CV death from baseline to end-of-trial (mean duration:
39.8 months) - subjects with chronic HF only

| | Time since randomisation to end-of-trial | | |
|---|---|---|---|
| | | | Two-sided |
| | Events (%) | 95% CI | p-value |
| Semaglutide 2.4 mg | 108 (5.0) | | |
| Placebo | 139 (6.5) | | |
| Treatment Hazard ratio | | | |
| Semaglutide 2.4 mg vs. placebo | 0.76 | (0.59-0.97) | 0.03 |

The results in Tables 19-20 show that patients treated with semaglutide achieved a reduction in heart failure leading to hospitalisation and CV death from baseline to end-of-trial compared with placebo.

The results in Tables 21-22 show that patients with chronic heart failure treated with semaglutide achieved a reduction in heart failure leading to hospitalisation and CV death from baseline to end-of-trial compared with placebo. Further, results in Table 22 show a statistically significant reduction by 24% for CV death in patients with chronic heart failure treated with semaglutide compared with placebo.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for the treatment of heart failure with preserved ejection fraction (HFpEF) in a human subject in need thereof, comprising administering semaglutide to the human subject in an amount of at least about 2.4 mg per week.

2. The method according to claim 1, wherein the human subject has a BMI of at least 27 kg/m².

3. The method according to claim 2, wherein the subject has a BMI of at least 30 kg/m².

4. The method according to claim 1, wherein the human subject has type 2 diabetes.

5. The method according to claim 1, wherein the human subject has a BMI of at least 27 kg/m² and type 2 diabetes.

6. The method according to claim 5, wherein the human subject has a BMI of at least 30 kg/m².

7. The method according to claim 1, wherein the semaglutide is administered once weekly.

8. The method according to claim 1, wherein the semaglutide is administered in an amount of 5-45 mg semaglutide once daily by oral administration.

9. The method according to claim 1, wherein the oral administration in the form of a solid composition comprising sodium N-(8-(2-hydroxybenzoyl)amino) caprylate (SNAC).

10. A method for preventing worsening of or improving heart failure with preserved ejection fraction (HFpEF) in a human subject in need thereof, comprising administrating semaglutide to the human subject in an amount of at least about 2.4 mg per week.

11. The method according to claim 10, wherein the human subject has a BMI of at least 27 kg/m².

12. The method according to claim 11, wherein the subject has a BMI of at least 30 kg/m².

13. The method according to claim 10, wherein the human subject has type 2 diabetes.

14. The method according to claim 10, wherein the human subject has a BMI of at least 27 kg/m² and type 2 diabetes.

15. The method according to claim 14, wherein the human subject has a BMI of at least 30 kg/m².

16. The method according to claim 10, wherein the semaglutide is administered once weekly.

17. The method according to claim 10, wherein the semaglutide is administered in an amount of 5-45 mg semaglutide once daily by oral administration.

18. The method according to claim 17, wherein the oral administration in the form of a solid composition comprising sodium N-(8-(2-hydroxybenzoyl)amino) caprylate (SNAC).
